(19) United States
(12) Patent Application Publication
(10) Pub. No.: US 2007/0203514 A1
(43) Pub. Date: Aug. 30, 2007

(54) Safe Locking Lancet

(75) Inventors: Joseph Flaherty, Westford, MA (US); Timothy Golnik, Boxford, MA (US)

Correspondence Address: Marina Larson & Associates, LLC, P.O. BOX 4928, DILLON, CO, 80435, US

(73) Assignee: AgaMatrix, Inc., Salem, NH (US)

(21) Appl. No.: 11/276,385

(22) Filed: Feb. 27, 2006

(51) Int. Cl. A61B 17/32

(52) U.S. Cl. 606/181

(57) ABSTRACT

A lancet is provided where the same opening of the same cap serves both as a removable cover prior to use of the lancet, and then interacts in a locking engagement with the lancet base to provide protection from contamination after use. Thus, the invention provides a lancet having a cutting portion and a cap portion. The cutting portion has a blade or needle, a base supporting the blade or needle, and a flange extending outward from the base. The cap portion (1) fits over the blade or needle and interacts with the base to provide a sealing engagement, (2) has a slot formed therein sized to slidably receive the flange, and (3) has a bound opening circumferentially displaced from the slot and sized to receive and capture the flange. The lancet may have a plurality of flanges extending from the base, for example two, and an equal plurality of slots and bound openings.

### BACKGROUND OF THE INVENTION

This application relates to a lancet of the type intended for single use to create a wound for the collection of a sample of blood or interstitial fluid. Lancets of this type are commonly used by diabetics to obtain samples for monitoring of glucose levels.

Conventional lancets have a base portion and a blade or needle. To maintain sterility of the lancet prior to use, a twist-off cap may be provided which is removed just prior to use by tearing a thin plastic connection between the cap and the lancet base. Such caps may, however, be small, and may not be adapted to cover the blade or needle after use. Thus, caps of this type do not reduce risk of contamination from blood on a used lancet that may arise from contact with the blood or accidental pricking with an exposed blade or needle.

Various patent relating to lancets disclose approaches for covering the lancet after use. U.S. Pat. No. 5,304,192 discloses a device with a removable cover and a larger case. Squeezing the case after use causes the case to deform to a position with the needle inside. U.S. Pat. No. 6,840,912 discloses a flip-cap that can be replaced over the needle after use. However, the cap is readily removable and this fails to allow the user to distinguish unequivocally between a used and an unused lancet. U.S. Pat. No. 5,207,699 shows a device with a two-ended cap and extractor. One end serves as a before-use cap. The other end has a plurality of fingers that serve to extract the lancet from the lancet firing device. U.S. Pat. No. 5,385,571 discloses a lancet cap that has a first opening for covering the needle tip prior to use, and a second larger opening for engaging with the lancet after use. Since there are two openings, however, there is the potential for ambiguity in the covering of the needle.

Notwithstanding these various cap styles, there remains room for improvement, particularly for providing a cap with few parts that is simple to make and unambiguous to use.

### SUMMARY OF THE INVENTION

In accordance with the present invention, a lancet is provided where the same opening of the same cap serves both as a removable cover prior to use of the lancet, and then interacts in a locking engagement with the lancet base to provide protection from contamination after use. Thus, the invention provides a lancet comprising a cutting portion and a cap portion,

wherein the cutting portion comprises a blade or needle, a base supporting the blade or needle, and a flange extending outward from the base, and

wherein the cap portion
(1) fits over the blade or needle and interacts with the base to provide a sealing engagement,
(2) has a slot formed therein sized to slidably receive the flange, and
(3) has a bound opening circumferentially displaced from the slot and sized to receive and capture the flange.

In specific embodiments, there are a plurality of flanges extending from the base, for example two, and an equal plurality of slots and bound openings.

### BRIEF DESCRIPTION OF THE DRAWINGS

FIGS. 1A and B show two embodiments of the cutting portion of a lancet of the invention from two angles.

FIG. 2 shows an embodiment of the invention in which the cap portion and the cutting portion are combined.

FIGS. 3A and 3B show an embodiment of the cap portion in accordance with the invention from two angles.

FIG. 4 shows a cross section through a combined cap portion 3 and cutting portion 1 in the radial plane of the bound opening 32.

FIGS. 5A and B show horizontal cross-sectional views through two cap portions in accordance with the invention.

FIGS. 6A-C show alternative shapes for flanges in accordance with the invention.

FIG. 7 shows a side view of a cutting portion with offset flanges.

FIGS. 8A and 8B show cap portions with variations in the bound opening.

### DETAILED DESCRIPTION OF THE INVENTION

The present application relates to an improved design for a lancet, and to a method of using such a lancet.

As used herein, the term “lancet” refers to a device with a cutting edge, in the form of a blade or needle, and a base that may be held by the user or that interacts with a device for triggering the cutting action of the lancet. Lancets are used to prick the skin (a finger, foot, ear lobe, etc.) to obtain a small quantity of capillary blood or interstitial fluid for testing.

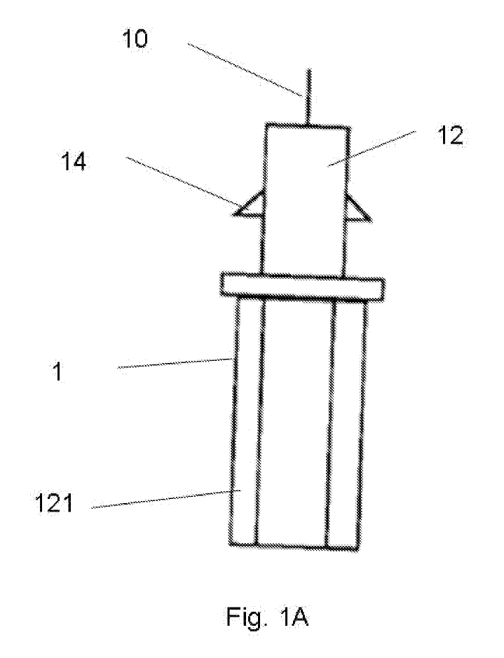

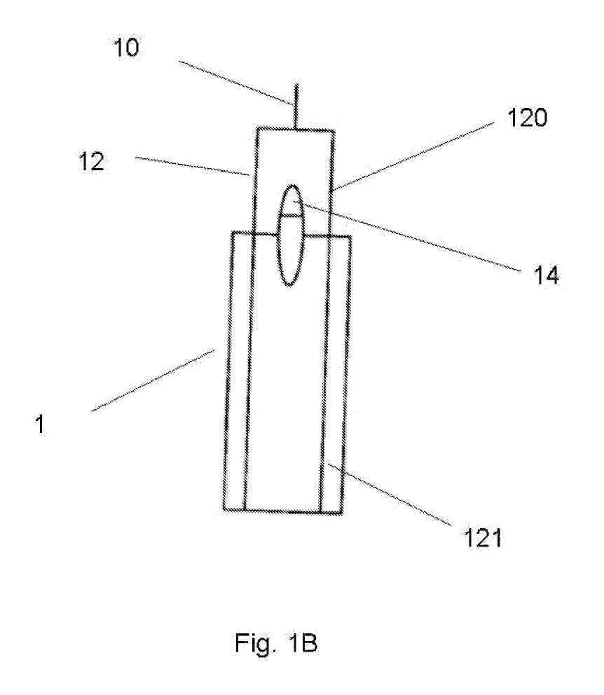

The lancet of the invention comprises a cutting portion and a cap portion. As shown in FIGS. 1A and 1 B, the cutting portion 1 comprises a blade or needle 10, a base 12 supporting the blade or needle 10, and a flange 14 extending outward from the base 12. The base 12 in FIGS. 1A and B includes a narrow portion 120 which is received within the cap, and an expanded portion 121 which remains exterior to the cap portion when it is placed on cutting portion, but the shape of these portions, and the use of separately defined portions are a matter of design choice as reflected in the differences between FIGS. 1A and 1B in this respect.

In the lancet of the invention, the cap portion fits over the blade or needle and interacts with the base to provide a sealing engagement. As used herein, the term “sealing engagement” means that the combination of the cap portion and the base interact, alone or in combination with an additional component, to provide a sealed space surrounding the needle or blade of the lancet. Desirably, the sealing engagement is one that maintains the sterility of the needle or blade prior to removal of the cap for use. By way of non-limiting example, this can occur as a result of a friction fit between the cap portion and the base, a snap interaction between the cap portion and the base, as a result of an additional part, such as a plastic overwrap or as a result of a combination thereof.

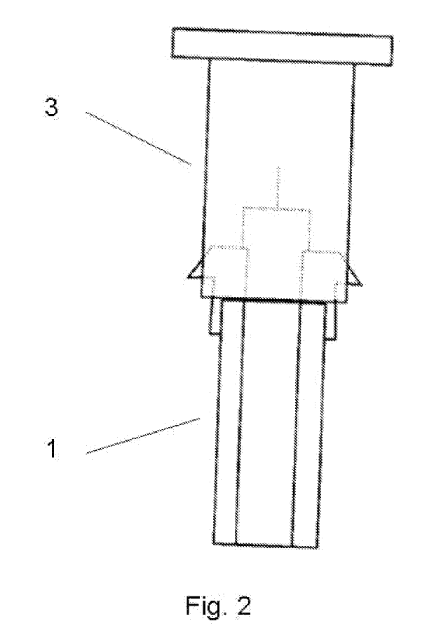

FIG. 2 shows an embodiment of the invention in which the cap portion 3 and the cutting portion 1 are combined. The parts of the cutting portion 1 within the cap portion 3 are shown in dotted lines, and the details of the cap portion 3 are omitted for clarity.

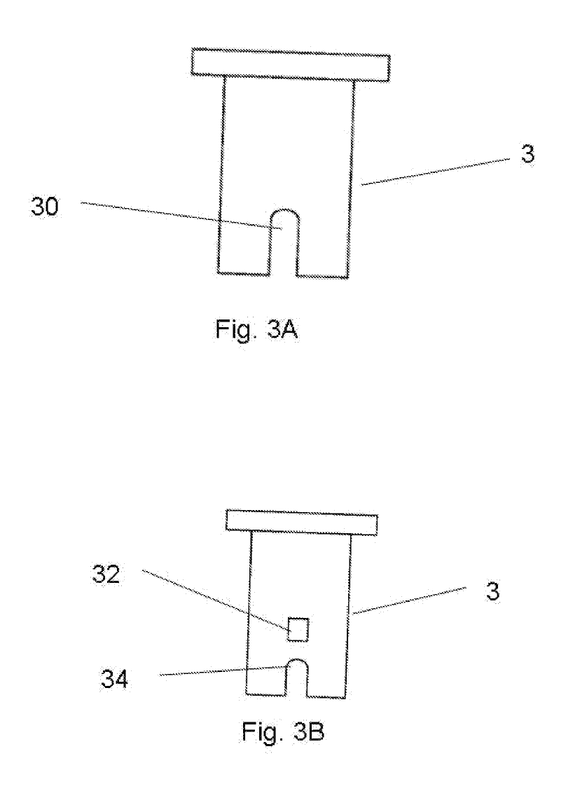

FIGS. 3A and 3B show an embodiment of the cap portion in accordance with the invention from two angles. As shown in FIG. 3A, the cap portion has a slot 30 formed therein. The slot 30 is sized to slidably receive a flange from a cutting portion inserted into the cap portion. Thus, the slot has a bottom width and a length into which the flange fits, and from which the flange can slide when the cap portion is removed for use. As used herein, the term “slidably received” means that the flange can be removed from the slot without application of force beyond that needed to overcome the sealing engagement of the cap portion with the cutting portion. FIG. 3B shows a different face of the same cap portion 3. On this face, there is a bound opening 32 sized to receive and capture the flange.

The embodiment of the invention shown in FIG. 3B also includes an alignment slot 34 in alignment with the bound opening 32. The flange interacts with the walls the cap portion 30, and the alignment slot, if present, and the flange is pressed inwards until the movement of the cap portion relative to the cutting portion brings the flange to the bound opening 30. At this point, the ends of the flange portion spring outwards through the bound opening, resulting in the capture of the cutting portion within the cap portion and substantially restricting removal of the cap portion.

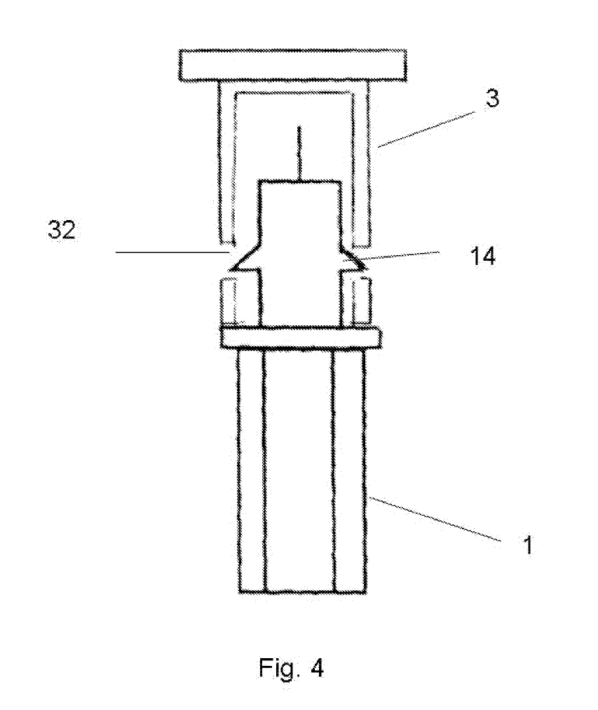

FIG. 4 shows a cross section through a combined cap portion 3 and cutting portion 1 in the radial plane of the bound opening 32, and more clearly shows the type of interaction that occurs when the flange is received and captured in the bound opening. As shown, the flanges 14 are extended through the bound opening 32. When the cap portion 3 is placed over a used Cutting portion 1, the flanges 14 are pressed downwards, until they come into alignment with the bound opening 32. At this point, the flanges 14 spring outwards through the bound opening 32 to the position shown. Efforts to pull the cutting portion 1 back out of the cap are resisted by the flanges 14 bearing on the lower edge of the bound opening 32 to result in a safety lock.

The embodiment shown in FIG. 4 does not have the alignment slot 34. This slot is optional, but is desirable because it aids the user in aligning the flange with the bound opening.

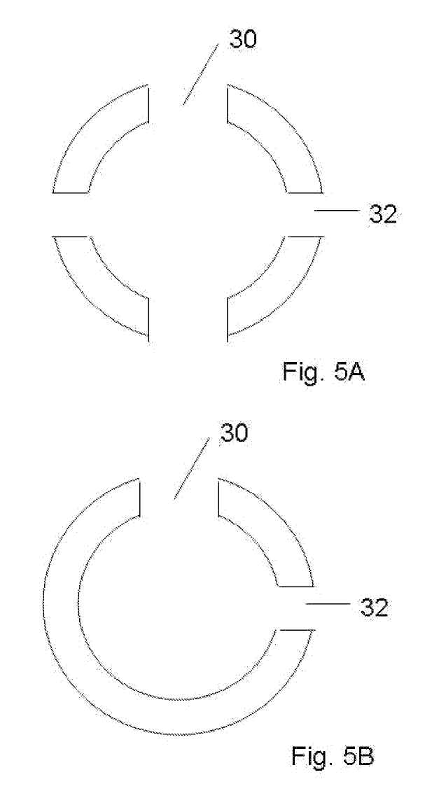

FIGS. 5A and B show horizontal cross-sectional views through two cap portion in accordance with the invention at a height passing through the bound opening 32 and the slot 30. In FIG. 5 A, there are two bound openings 32 disposed opposite on another, and two slots 30 disposed opposite one another. The slots 30 are circumferentially displaced by 90 degrees from the bound openings 32. In FIG. 5B an embodiment is shown in which there is only one slot 30, and one bound opening 32, circumferentially displaced by 90 degrees. The use of 90 degree displacement is convenient, but it is not required. Further, additional slots and bound openings could be provided if desired. It will be appreciated that while FIGS. 5A and B show the cap portion as circular in cross section, that this shape is not required, provided that the cap has sufficient symmetry to allow flanges on the base to interact with either the slot(s) or the bound openings(s). Such symmetry can be obtained in the case of one flange in any shape have a mirror plane of symmetry such as an oval, rectangle or ellipse, or using an equilateral triangle. If a plurality of flanges are used, the cross-sectional shape of the cap can be circular or a regular equilateral polygon, such as a square, pentagon or hexagon.

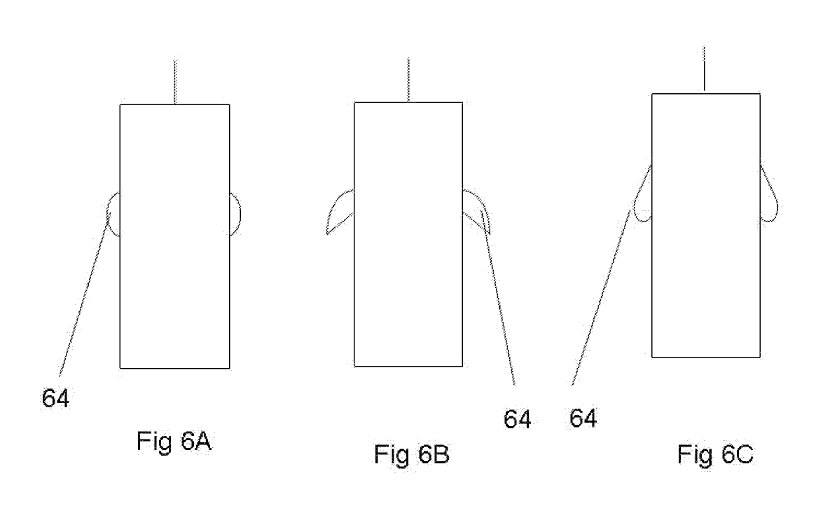

FIGS. 6 A-C B show several alternative shapes for the flange portions. As shown in FIG. 6A, the flange 64 has a semicircular appearance in profile view. The flange 64 as shown in FIG. 6B has two distinct surfaces. The upper surfaces is smooth and has a convex curvature to facilitate movement into the cap portion prior to achieving a sealing engagement. The lower surface is generally flat to engage and latch within the bound opening. In FIG. 6C, the end of the flange is rounded, buy it has a downward angle to restrict removal of the cap once in place to a great extent that the flange of FIG. 6A.

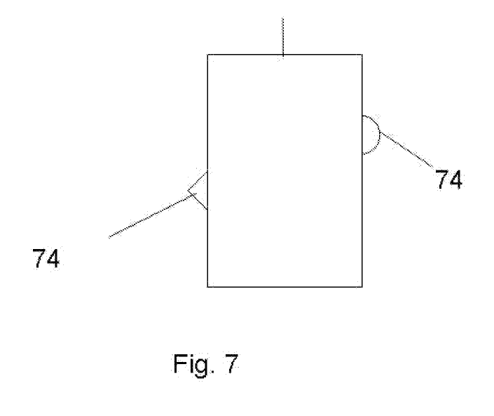

As shown in FIG. 4, the number of flanges may suitably be equal to the number of slots and to the number of bound openings. In a general sense, where there are a plurality of flanges, it is preferred that there by an equal plurality, that is the same number of slots and an equal plurality of bound openings distributed at angular intervals around the cap portion that correspond to the distribution flanges around the base of the cutting portion as this balances locking action of the flanges. When the cutting portions has a plurality of flanges, the flanges may be disposed such that the cutting portion has rotational symmetry. Thus, as shown in FIG. 1A, the two flanges 14 extend outward from circumferentially opposite locations relative to a center axis of the cutting portion 12. Alternatively, the flanges may be disposed such that there is no rotational symmetry. In one non-symmetrical embodiment, the flanges are disposed at a common height, but are not disposed in circumferentially opposite positions. In another embodiment, as shown in FIG. 7, the flanges 74, 74′ are displaced in height relative to one another. In FIG. 7, the flanges 74 and 74′ also have different shapes, but this is not critical, providing that the flanges interact with the cap portion to capture the flanges and retain the cap portion on the used lancet. A lancet may also have flanges displaced both in height and circumferentially. It will be appreciated, however, that symmetry is desirable as it makes putting the cap on the lancet easier.

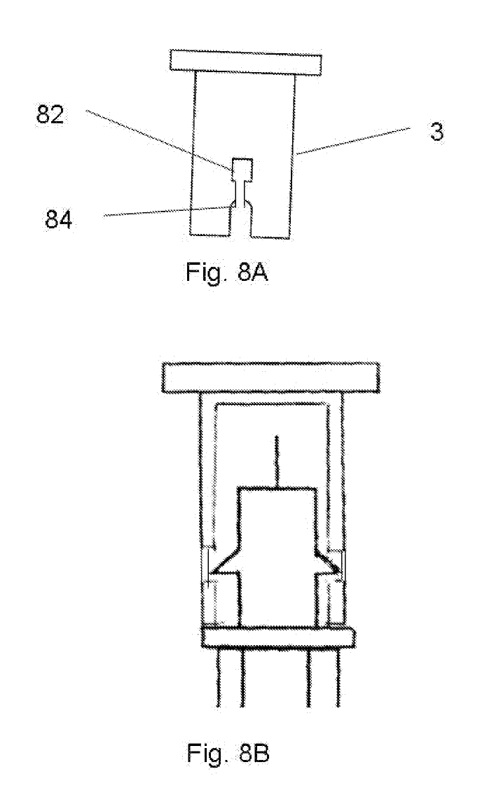

Although the bound opening discussed above is fully closed on the bottom and open to the exterior of the cap, neither of these is required for the usefulness of the invention. For example, as shown in FIG. 8A, the bound opening 82 may be partially open at the bottom and in communication with an alignment slot 84, provided that extent to which it is open at the bottom does not allow free movement of the flange downward and out of the cap. The bound opening may also closed or partially close relative to the exterior surface of the cap portion, such that it is a depression formed on the interior of the cap portion as depicted in FIG. 8B.

1. A lancet comprising
a cutting portion and a cap portion,
wherein the cutting portion comprises a blade or needle, a base supporting the blade or needle, and a flange extending outward from the base, and
wherein the cap portion
(1) fits over the blade or needle and interacts with the base to provide a sealing engagement,
(2) has a slot formed therein sized to slidably receive the flange, and
(3) has a bound opening circumferentially displaced from the slot and sized to receive and capture the flange.

2. The lancet of claim 1, wherein there are a plurality of flanges extending from the base, and a equal plurality of slots and bound openings.

3. The lancet of claim 2, wherein there are two flanges extending from the base.

4. The lancet of claim 2, wherein the two flanges are disposed circumferentially opposite to each other.

5. The lancet of claim 1, wherein slot and bound opening are displaced by 90 degrees relative to one another.
